(19) United States
(12) Patent Application Publication
(10) Pub. No.: US 2010/0086993 A1
(43) Pub. Date: Apr. 8, 2010

(54) CELL DETECTING SYSTEM AND QUANTUM DOT MEASURING SYSTEM

(75) Inventors: LEE-JENE LAI, HSIN-CHU (TW); YI-HEUI HSIEH, HSIN-CHU (TW); SHIH-JEN LIU, MIAOLI COUNTY (TW); HSIN-WEI CHEN, MIAOLI COUNTY (TW)

Correspondence Address: ROSENBERG, KLEIN & LEE, 3458 ELLICOTT CENTER DRIVE-SUITE 101, ELLICOTT CITY, MD, 21043, US

(73) Assignee: NATIONAL SYNCHROTRON RADIATION RESEARCH CENTER, HSIN-CHU (TW)

(21) Appl. No.: 12/272,130

(22) Filed: Nov. 17, 2008

(30) Foreign Application Priority Data
Oct. 7, 2008 (TW) 97138557

(51) Int. Cl. C12M 1/42; G01J 1/42

(52) U.S. Cl. 435/287.2; 435/288.7; 250/361 R; 977/925

(57) ABSTRACT

A cell detecting system is herein disclosed, wherein a complex is formed with a first bioactive ligand coupling to a quantum dot and recognizing and coupling to a first receptor of a cell and a second bioactive ligand coupling to a magnetic bead and recognizing and coupling to a second receptor of the cell. The magnet is configured for attracting the complex. A quantum dot measuring system configured for measuring the fluorescence of the complex includes an excitation light source, an optical system, a detecting sensor and a data capturing unit, wherein the detecting sensor includes a photomultiplier tube measuring florescence of the quantum dot excited by the excitation light source. The present invention achieves the goal of specific cell detection with high sensitivity without performing cell incubation. A quantum dot measuring system is also herein disclosed.

### BACKGROUND OF THE INVENTION

1. Field of the Invention

The present invention relates to a cell detecting system and quantum dot measuring system, more particularly to a cell detecting system using a magnetic bead, a quantum dot and a quantum dot measuring system with a photomultiplier tube.

2. Description of the Prior Art

Many diseases are caused by pathologic cells. For example, the mad cow disease is a neuronal pathology caused by abnormally folded prion, infective to different animals and incurable for now. A cervical cancer is caused by pathologic epidermal cells in cervix uterus. The number of pathologic cells is little in early stage; therefore, it is very important to improve the sensitivity and detection time for specific cells detection in the presence of trace pathologic cells.

The most commonly practiced methods in specific cell detection include immunofluorescence analysis and flow cytometry for now. The immunofluorescence analysis includes staining and then performing microscopic examination and counting with fluorescence microscope. Therefore, the immunofluorescence analysis includes the drawback of being time-consuming, labor-consuming and error-prone.

A flow cytometer is likely unable to analyze low amount of specific cells (less than 0.01%), due to the low signal to noise ratio. Therefore, it is necessary to perform cell culture to increase the cell amount for flow cytometry. It takes a lot of time for cell culture, and the cell detection is thus unable to be performed in a time-effective manner. In addition, most of the fluorescent markers used in the above-mentioned specific cell detection methods are organic fluorescent markers which rapidly decay when illuminated with ultraviolet light and cause the difficulty in counting cells.

In sight of the drawbacks of organic fluorescent markers, fluorescence markers of high fluorescence and stability have been researched by scientists. An inorganic quantum dot is first reported in 1998 to couple to cells or protein molecules with 20 folds greater in luminance in fluorescence microscope compared to the organic fluorescent markers.

Specific cells usually exist in the mixture of cells and are not easily specifically detected. Therefore, it is necessary to couple the inorganic quantum dot to the specific cells and isolate specific cells with the inorganic quantum dot from the mixture of cells.

Methods for isolating cells include centrifuge, column chromatography, flow cytometry and magnetic bead isolation. The magnetic bead isolation takes advantage of magnetism; that is to say magnetic beads are attracted in a magnetic field and are free and mobile in a non-magnetic field. A specific antibody is connected to the surface of a magnetic bead and then couples to the antigen on the surface of the cell for specific cells. The specific cell, which is connected to the surface of the magnetic bead, is isolated under the magnet. Magnetic bead isolation includes advantages of high specificity, simple operation and low cost by coupling of antibody and antigen.

Su et al (Anal. Chem. 76, 4806, 2004) adopted a quantum dot coupled with immuno-magnetic separation for detection of Salmonella and Escherichia coli O157:H7. However, the detecting sensor for Su et al adopted is a CCD (Charge-coupled device) which has limitation in detection sensitivity. In addition, bacteria are likely to form colonies and pathological cells in human bodies, which are shedding cells; therefore, the sensitivity requirement for detecting pathological cells is higher than detecting bacteria colonies.

To sum up, it is now a current goal to adopt a magnetic bead and quantum dot to achieve specific cell detection of high sensitivity without performing cell culture.

### SUMMARY OF THE INVENTION

A cell detecting system is provided to use a magnetic bead, a quantum dot and a quantum dot measuring system with a photomultiplier tube and to achieve the goal of specific cell detection with high sensitivity without performing cell incubation.

A quantum dot measuring system is provided to use a photomultiplier tube and to achieve quantum dot measuring with high sensitivity.

In one embodiment, the proposed cell detecting system includes a quantum dot; a first bioactive ligand coupling to the quantum dot, wherein the first bioactive ligand recognizes and couples to a first receptor of a cell; a magnetic bead; a second bioactive ligand coupling to the magnetic bead, wherein the second bioactive ligand recognizes and couples to a second receptor of the cell, and a complex is formed with the first bioactive ligand, the quantum dot, the second bioactive ligand, the magnetic bead and the cell; a magnet configured for attracting the complex; and a quantum dot measuring system including an excitation light source configured for providing an exciting energy for the quantum dot of the complex to emit fluorescence; a detecting sensor configured for detecting the fluorescence, wherein the detecting sensor includes a photomultiplier tube converting the fluorescence into a signal; an optical system relaying the fluorescence to the detecting sensor; and a data capturing unit electrically connected to the detecting sensor and capturing the signal.

In another embodiment, the proposed quantum dot measuring system includes an excitation light source, a detecting sensor, an optical system, and a data capturing unit. The excitation light source is configured for providing an exciting energy for a quantum dot to emit fluorescence; the detecting sensor configured for detecting the fluorescence, wherein the detecting sensor comprises a photomultiplier tube converting the fluorescence into a signal; the optical system relays the fluorescence to the detecting sensor; and the data capturing unit electrically connected to the detecting sensor and capturing the signal.

Other advantages of the present invention will become apparent from the following description taken in conjunction with the accompanying drawings wherein are set forth, by way of illustration and example, certain embodiments of the present invention.

### BRIEF DESCRIPTION OF THE DRAWINGS

The foregoing aspects and many of the accompanying advantages of this invention will become more readily appreciated as the same becomes better understood by reference to the following detailed description, when taken in conjunction with the accompanying drawings, wherein:

FIG. 1 is a schematic view diagram illustrating a cell detecting system according one preferred embodiment of the present invention;

FIG. 2 is a schematic view diagram illustrating an embodiment of the present invention;

FIG. 3 is a schematic view diagram illustrating a quantum dot measuring system according to an embodiment of the present invention;

FIG. 4a is a schematic view of the experimental outcome of an embodiment of the present invention; and

FIG. 4b is a schematic view of the experimental outcome of an embodiment of the present invention.

### DESCRIPTION OF THE PREFERRED EMBODIMENT

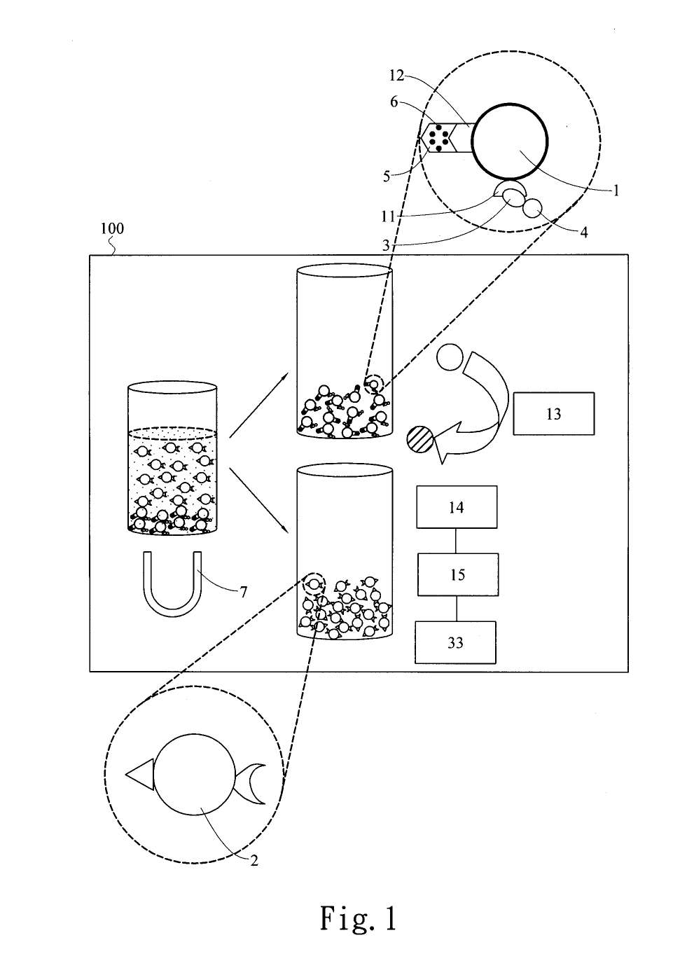

Referring to FIG. 1, a cell detecting system 100 according to an embodiment of the present invention is provided. The trapping and detecting theory is firstly disclosed. In this embodiment, a specific cell 1 includes a first receptor 11 and a second receptor 12. A first bioactive ligand 3 is coupled to a quantum dot 4 and is capable of recognizing and coupling to the first receptor 11 of the specific cell 1; and a second bioactive ligand 5 is coupled to a magnetic bead 6 and is capable of recognizing and coupling to the second receptor 12 of the specific cell 1, wherein the diameter of the magnetic bead 6 is between 25 nm and 5000 nm.

With the above-mentioned coupling mechanism, a complex is formed with the specific cell 1, first bioactive ligand 3, quantum dot 4, second bioactive ligand 5 and magnetic bead 6 while a second cell 2 which is lack of the first receptor 11 and second receptor 12 is not recognized by and coupled to the first bioactive ligand 3 and second bioactive ligand 5 and it is thus unable to form such a complex. Therefore, the above-mentioned configuration achieves the goal of isolating cells. The complex with the magnetic bead 6 may be further attracted by a magnet 7 for cell trapping. In addition, the complex having quantum dot 4 which is of high fluorescence and stability may be applied for high-sensitivity detection.

The first bioactive ligand 3 and the second bioactive ligand 5 respectively comprise an antibody, a small molecule, a nucleotide, or a protein assembly. Here, the small molecule, for example, is a pentazocine, an anisamide, or a haloperidol coupling to a sigma receptor on the cell. A nucleotide, e.g. deoxyribonucleic acid (DNA) and ribonucleic acid (RNA), forms an aptomer which recognizes a specific receptor. In addition, the protein assembly includes a major histocompatibility complex and a peptide, and the protein assembly specifically recognizes a T cell receptor.

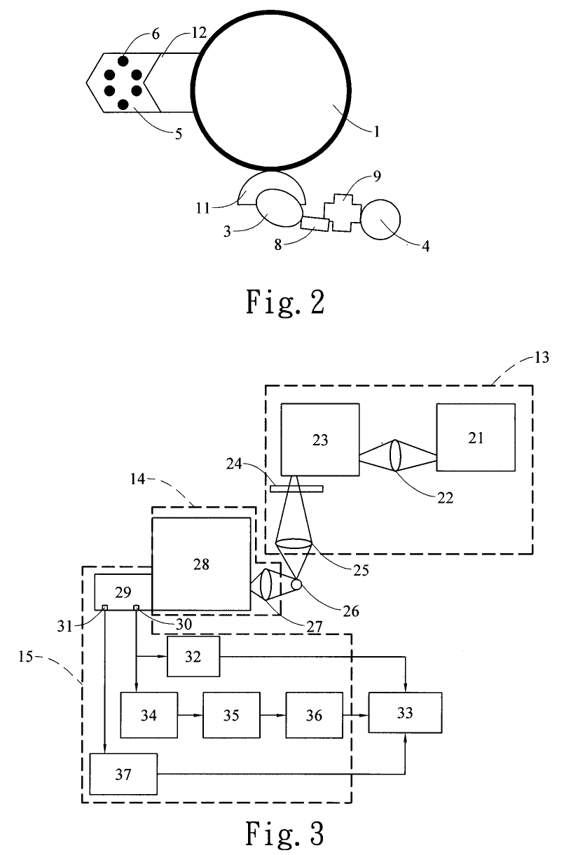

The coupling of the first bioactive ligand 3 to the quantum dot 4 and the second bioactive ligand 5 to the magnetic bead 6 may be direct or indirect. FIG. 2 illustrates an example in which the first bioactive ligand 3 is indirectly coupled to the quantum dot 4. For example, the first bioactive ligand 3 is indirectly coupled to the quantum dot 4 with a biotin 8 and a streptavidin 9. The coupling of the biotin 8 and streptavidin 9 is of high association constant (10¹⁴ M⁻¹) with four biotins coupling to a streptavidin and is commonly practiced in coupling between biological molecules.

In one example, the quantum dot 4 includes a PbS quantum dot, a II-VI quantum dot, or a III-V quantum dot. The II-VI quantum dot includes a CdSe quantum dot or a CdTe quantum dot, wherein the II-VI quantum dot may be encapsulated with a ZnS coating. The III-V quantum dot includes an InP quantum dot, a GaN quantum dot, or an InAs quantum dot encapsulated with a GaAs coating.

The fluorescence measuring system of the present invention is next described. Referring to FIG. 1, a quantum dot measuring system according to one preferred embodiment of the present invention includes an excitation light source 13, a detecting sensor 15, an optical system 14 and a data capturing unit 33. The excitation light source 13 is configured for providing an exciting energy for the quantum dot 4 of the complex to emit fluorescence. The optical system 14 relays the fluorescence to the detecting sensor 15 configured for detecting the fluorescence. The signal measured by the detecting sensor 15 is then transmitted to the data capturing unit 33 electrically connected to the detecting sensor 15 and capturing the signal. Here, the detecting sensor 15 includes a photomultiplier tube converting the fluorescence into a signal to improve the detection sensitivity of the quantum dot measuring system.

Refer to FIG. 1 and FIG. 3 for further detailed description, in which FIG. 3 illustrates a quantum dot measuring system according to an embodiment of the present invention. The excitation light source 13 including a light source 21, a first lens 22, a first monochromator 23, a spill shield 24 and a second lens 25 excites a cell sample 26 having the quantum dot. The example of light source 21 may include an ultraviolet light, a light-emitting diode, or a laser light. In addition, the pH value is between 9 and 14 in the incubation environment of the quantum dot measuring system.

The excited fluorescence is relayed by the optical system 14 including a third lens 27 and a second monochromator 28 to the detecting sensor 15 which includes a photomultiplier tube 29.

The signal measured by the photomultiplier tube 29 is further converted into a current signal or a pulse signal then captured by the data capturing unit 33. In one embodiment, the photomultiplier tube 29 includes a current mode 30 used for converting the signal measured by the photomultiplier tube 29 into a current signal, and the detecting sensor 15 further includes an ammeter 32 electrically connected to the current mode 30 and measuring the current signal. In another embodiment, in addition, the detecting sensor 15 further includes a lock-in amplifier 34, a voltage-to-frequency converter 35 and a frequency counter 36. Here, the lock-in amplifier 34 is electrically connected to the current mode 30 and converts the current signal into a voltage output; the voltage-to-frequency converter 35 is electrically connected to the lock-in amplifier 34 and converts the voltage generated by the lock-in amplifier 34 into frequency then output by the frequency counter 36 to the data capturing unit 33.

In another embodiment, the photomultiplier tube 29 includes a pulse mode 31 used for converting the signal measured by the photomultiplier tube 29 into a pulse signal. The pulse signal generated by the photomultiplier tube 29 is transmitted to the photon counter 37 which is electrically connected to the pulse mode 31 and output to the data capturing unit 33.

Furthermore, it is noted that the photomultiplier tube is cooled in a vacuumed or non-vacuumed way to lower the background current of the photomultiplier tube in one embodiment of the present invention, and the temperature of the photomultiplier tube is between −200° and 25°.

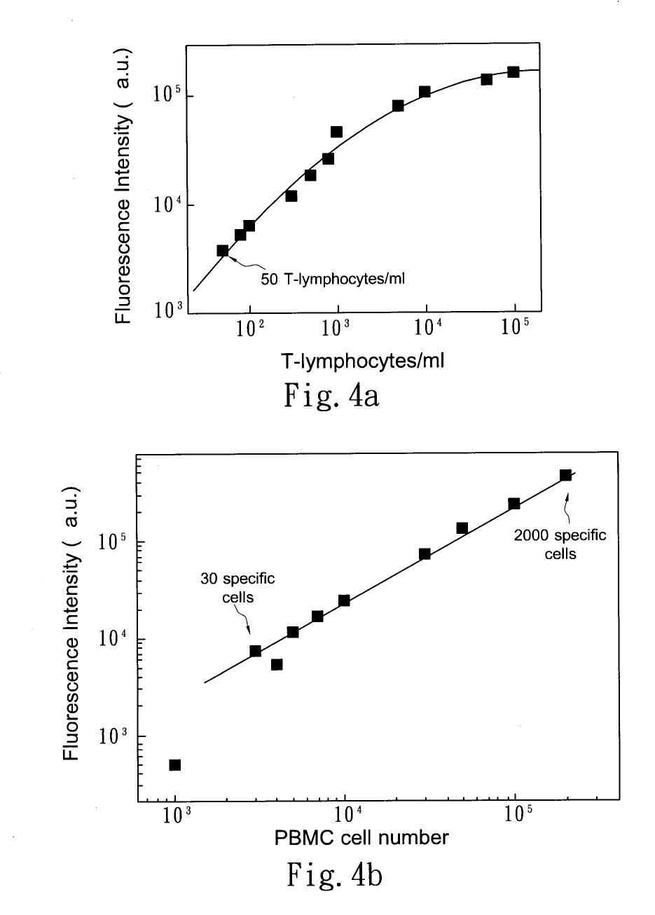

Referring to FIG. 2, in an embodiment, the specific cells 1 are human T-lymphocytes having a first receptor 11 and a second receptor 12, e.g. a CD3 or CD4 marker. Second cells 2, e.g. B-lymphocytes, having a CD19 or CD40 marker on their surfaces are mixed into the environment where the T-lymphocytes are incubated. In this embodiment, the T-lymphocytes and B-lymphocytes are well mixed. The first bioactive ligand 3, i.e. an anti-CD3 antibody, reacts with the CD3 on the T-lymphocytes and then couples to the quantum dot 4 by biotin 8 and streptavidin 9, and the T-lymphocytes are thus coupled with the quantum dot 4. The CD4 marker of the T-lymphocytes is then coupled to a second bioactive ligand 5, i.e. an anti-CD4 antibody, with a magnetic bead 6 to form a complex. Referring to FIG. 4a shows the experimental outcome according to this embodiment, the detection sensitivity of the experiment is, but not limited to, about 50 specific cells/ml in total of 10⁶ mixing cells/ml.

Another embodiment of the present invention includes a method for detecting the percentage of human PBMC (peripheral blood mononuclear cell) containing EB (Epstein-Barr) virus. In this embodiment, the first bioactive ligand includes a MHC (Major histocompatibility complex) bonded with a specific EB virus peptide (SSCSSCPLSK) monomer for specifically recognizing T-cell receptor. The EB virus peptide specifically recognizes the first receptor of the EB virus specific T-cell, e.g. a T-cell receptor of EB virus containing cells. The MHC monomer couples to a biotin to form a MHC-peptide-biotin which further couples to a streptavidin with a quantum dot to form a multimer. A second receptor, e.g. a CD8 marker, on the PBMC cell surface is then coupled by a second bioactive ligand, i.e. an anti-CD8 antibody, with a magnetic bead to form a complex. The specific cells are isolated and then applied for quantum dot fluorescence measuring. Referring to FIG. 4b shows the experimental outcome of this embodiment. There are 1% EB virus containing cells and the detection limits is about 3000 PBMC, i.e. 30 EB virus containing cells. The sensitivity of the present embodiment is 0.003% and is much better than 0.01% for conventional flow cytometry sensitivity based on the presumption of 10⁶ cells/ml in the blood.

To sum up, a cell detecting system according to the present invention using a magnetic bead, a quantum dot and a quantum dot measuring system with a photomultiplier tube achieves the goal of specific cell detection with high sensitivity without performing cell incubation.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the spirit and scope of the appended claims.

What is claimed is:

1. A cell detecting system, comprising:
a quantum dot;
a first bioactive ligand coupling to the quantum dot, wherein the first bioactive ligand recognizes and couples to a first receptor of a cell;
a magnetic bead;
a second bioactive ligand coupling to the magnetic bead, wherein the second bioactive ligand recognizes and couples to a second receptor of the cell, and a complex is formed with the first bioactive ligand, the quantum dot, the second bioactive ligand, the magnetic bead and the cell;
a magnet configured for attracting the complex; and
a quantum dot measuring system comprising:
an excitation light source configured for providing an exciting energy for the quantum dot of the complex to emit fluorescence;
a detecting sensor configured for detecting the fluorescence, wherein the detecting sensor comprises a photomultiplier tube converting the fluorescence into a signal;
an optical system relaying the fluorescence to the detecting sensor; and
a data capturing unit electrically connected to the detecting sensor and capturing the signal.

2. The cell detecting system as claimed in claim 1, wherein the quantum dot comprises a PbS quantum dot, a II-VI quantum dot or a III-V quantum dot.

3. The cell detecting system as claimed in claim 2, wherein the II-VI quantum dot comprises a CdSe quantum dot or a CdTe quantum dot.

4. The cell detecting system as claimed in claim 2, wherein the II-VI quantum dot is encapsulated with a ZnS coating.

5. The cell detecting system as claimed in claim 2, wherein the III-V quantum dot comprises an InP quantum dot, a GaN quantum dot, or an InAs quantum dot encapsulated with a GaAs coating.

6. The cell detecting system as claimed in claim 1, wherein the quantum dot and the first bioactive ligand is coupled by a streptavidin and a biotin.

7. The cell detecting system as claimed in claim 1, wherein the magnetic bead and the second bioactive ligand is coupled by a streptavidin and a biotin.

8. The cell detecting system as claimed in claim 1, wherein the first bioactive ligand and the second bioactive ligand respectively comprise an antibody, a small molecule, a nucleotide or a protein assembly.

9. The cell detecting system as claimed in claim 8, wherein the protein assembly comprises a MHC (major histocompatibility complex) and a peptide, and the protein assembly specifically recognizes a T cell receptor.

10. The cell detecting system as claimed in claim 1, wherein the diameter of the magnetic bead is between 25 nm and 5000 nm.

11. The cell detecting system as claimed in claim 1, wherein the pH value is between 9 and 14 in the incubation environment of the quantum dot measuring system.

12. The cell detecting system as claimed in claim 1, wherein the excitation light source comprises an ultraviolet light, a light-emitting diode or a laser light.

13. The cell detecting system as claimed in claim 1, wherein the photomultiplier tube is cooled in a vacuumed or non-vacuumed way to lower the background current of the photomultiplier tube.

14. The cell detecting system as claimed in claim 1, wherein the temperature of the photomultiplier tube is between −200° C. and 25° C.

15. The cell detecting system as claimed in claim 1, wherein the photomultiplier tube comprises a pulse mode used for converting the signal measured by the photomultiplier tube into a pulse signal.

16. The cell detecting system as claimed in claim 15, wherein the detecting sensor further comprises a photon counter electrically connected to the pulse mode.

17. The cell detecting system as claimed in claim 1, wherein the photomultiplier tube comprises a current mode used for converting the signal measured by the photomultiplier tube into a current signal.

18. The cell detecting system as claimed in claim 17, wherein the detecting sensor further comprises an ammeter electrically connected to the current mode.

19. The cell detecting system as claimed in claim 18, the detecting sensor further comprises a lock-in amplifier, a voltage-to-frequency converter and a frequency counter electrically connected to the current mode in series.

20. A quantum dot measuring system comprising:
an excitation light source configured for providing an exciting energy for a quantum dot to emit fluorescence;
a detecting sensor configured for detecting the fluorescence, wherein the detecting sensor comprises a photomultiplier tube converting the fluorescence into a signal;
an optical system relaying the fluorescence to the detecting sensor; and
a data capturing unit electrically connected to the detecting sensor and capturing the signal.

21. The quantum dot measuring system as claimed in claim 20, wherein the quantum dot comprises a PbS quantum dot, a II-VI quantum dot or a III-V quantum dot.

22. The quantum dot measuring system as claimed in claim 21, wherein the II-VI quantum dot comprises a CdSe quantum dot or a CdTe quantum dot.

23. The quantum dot measuring system as claimed in claim 21, wherein the II-VI quantum dot is encapsulated with a ZnS coating.

24. The quantum dot measuring system as claimed in claim 21, wherein the III-V quantum dot comprises an InP quantum dot, a GaN quantum dot, or an InAs quantum dot encapsulated with a GaAs coating.

25. The quantum dot measuring system as claimed in claim 20, wherein the excitation light source comprises an ultraviolet light, a light-emitting diode or a laser light.

26. The quantum dot measuring system as claimed in claim 20, wherein the photomultiplier tube is cooled in a vacuumed or non-vacuumed way to lower the background current of the photomultiplier tube.

27. The quantum dot measuring system as claimed in claim 20, wherein the temperature of the photomultiplier tube is between −200° C. and 25° C.

28. The quantum dot measuring system as claimed in claim 20, wherein the photomultiplier tube comprises a pulse mode used for converting the signal measured by the photomultiplier tube into a pulse signal.

29. The quantum dot measuring system as claimed in claim 28, wherein the detecting sensor further comprises a photon counter electrically connected to the pulse mode.

30. The quantum dot measuring system as claimed in claim 20, wherein the photomultiplier tube comprises a current mode used for converting the signal measured by the photomultiplier tube into a current signal.

31. The quantum dot measuring system as claimed in claim 30, wherein the detecting sensor further comprises an ammeter electrically connected to the current mode.

32. The quantum dot measuring system as claimed in claim 31, the detecting sensor further comprises a lock-in amplifier, a voltage-to-frequency converter and a frequency counter electrically connected to the current mode in series.
